(19) United States
(12) Patent Application Publication
(10) Pub. No.: US 2022/0354950 A1
(43) Pub. Date: Nov. 10, 2022

(54) TREATMENT AND CONTROL OF BACTERIAL AND FUNGAL DISEASE IN HONEYBEES

(71) Applicant: Tallgrass Biologics L.L.C., Worthington, MN (US)

(72) Inventor: Ben Hause, Slayton, MN (US)

(21) Appl. No.: 17/771,175

(22) PCT Filed: Oct. 22, 2020
(86) PCT No.: PCT/US2020/056755

§ 371 Date: Apr. 22, 2022

Related U.S. Application Data
(60) Provisional application No. 62/924,503, filed on Oct. 22, 2019.

(51) Int. Cl. A61K 39/40; A61K 9/00; A61K 39/395; A61K 35/57; A61P 31/04; A23K 20/163; A23K 20/195; A23K 50/90

(52) U.S. Cl. CPC A61K 39/40; A61K 9/0056; A61K 39/39575; A61K 35/57; A61P 31/04; A23K 20/163; A23K 20/195; A23K 50/90

(57) ABSTRACT

A prophylactic and/or therapeutic food composition for control of bacterial and/or fungal infection in bees and/or bee larvae, and methods of using and making the same. Bee feed compositions comprising bacterial and/or fungal antibodies dispersed in an edible base composition. Use of bacterial and/or fungal antigenic agents to passively generate bacterial and/or fungal antibodies in chicken egg yolks, and use of such bacterial and/or fungal antibodies for prophylaxis or treatment of bacterial and/or fungal infection in the bees and/or larvae.

### CROSS-REFERENCE TO RELATED APPLICATIONS

The present application claims the priority benefit of U.S. Provisional Patent Application Ser. No. 62/924,503, filed Oct. 22, 2019, entitled TREATMENT AND CONTROL OF BACTERIAL AND FUNGAL DISEASE IN HONEYBEES, incorporated by reference in its entirety herein.

### BACKGROUND OF THE INVENTION

### Field of the Invention

The present invention relates to the use of antibodies against bacterial and/or fungal disease for prophylaxis and/or treatment of honeybees.

### Description of Related Art

Worldwide, honeybee (Apis mellifera) colonies have seen dramatic population losses, a phenomenon called colony collapse disorder (CCD). Numerous causative factors have been investigated, including loss of habitat and plant diversity, increasing use of insecticides and several bee pathogens, including bacteria, fungi, mites, and viruses. The spread of CCD across the globe has correlated with the introduction of the bee parasitic mite Varroa destructor. For example, the introduction of V. destructor on the Hawaiian Islands in the early 2000's coincided with first reports of CCD on the islands. While the Varroa mites feed on both pupa and adult bees, they have been eliminated as a direct cause of CCD as mite control measures utilized in collapsing colonies minimize mite colonization, yet fail to prevent CCD.

Current CCD prophylaxis and treatment focus on control of Varroa. While these efforts are often helpful, increased efficacy is necessary. Unlike vertebrates which have both innate and adaptive immunity, insects possess only innate immunity. The insect immune system can recognize conserved pathogen features via pattern recognition receptors which activates general anti-pathogen defenses. The insect genomes however lack the ability to mount a specific, pathogen-specific immune response. In vertebrates, activation of the adaptive immune system employs somatic cell recombination in B and T lymphocytes which gives rise to highly specific antibody and cell-based responses to the pathogen. Insects lack this ability.

Vertebrates are often born with an immune system that is not fully mature and consequently lacks the ability to generate adaptive immune responses. To overcome this deficiency, maternal antibodies are delivered to the newborn to provide passive protection while the immune system matures. In avian species, maternal antibodies can be found in the egg yolk. In mammals, maternal antibodies are passed to the newborn via colostrum and nursing. These antibodies are referred to as passive as they are formed by the mother and transferred to the newborn. In mammals, these antibodies typically have a half-life of approximately two weeks and often provide protection from the particular pathogen for the first month or two of life. Passive antibodies have been exploited for the protection of animal health. For example, vaccination of pregnant females is utilized to generate an immune response which will be transferred to neonates via nursing. In biotechnology, animals are often hyperimmunized with a pathogen antigen to generate high antibody titers. Antibodies are subsequently purified from the hyperimmunized animal sera and injected into susceptible or infected animals. Likewise, antibodies are purified from hyperimmunized animal colostrum or eggs and used to feed susceptible or infected neonates to provide protection. Recently, researchers in China hyperimmunized chickens using whole inactivated Sacbrood virus and isolated antibodies from egg yolk (Sun et al., Preparation and application of egg yolk antibodies against Chinese Sacbrood virus infection. Frontiers Microbiology 9:1814, 2018). Bees feed anti-Sacbrood virus antibodies were protected from disease caused by Sacbrood virus. The use of antibodies in bees is unexpected, and it is unknown whether similar approaches would work for other pathogens.

### SUMMARY OF THE INVENTION

The present invention is broadly concerned with the passive generation of antibodies against bacterial and/or fungal (particularly parasitic fungal) infection in chickens (“chicken- or egg-derived antibodies”) and their use for antibody prophylaxis and therapeutics in honeybee colonies. In one or more embodiments, the antibodies are immunoglobulin Y antibodies (IgY). In one or more embodiments, the antibodies are derived from (i.e., purified, separated, etc.) egg yolk. In one or more embodiments, the antibodies are from hens immunized with a specific antigen (e.g., bacterial and/or fungal antigenic agent) to produce a specific IgY in the egg yolk against the given antigen. Embodiments of the invention also involved immunizing hens with a bacterial or fungal agent to generate such antibodies.

In one or more embodiments, the invention is concerned with prophylactic and/or therapeutic bee (or larvae) feed or supplements comprising anti-bacterial and/or anti-fungal antibodies, preferably dispersed in an edible base composition. The edible base composition typically comprises a carbohydrate source or is formulated for mixing with a carbohydrate source. The antibodies can be used in liquid solution or as lyophilized dry form with commercial bee feed or feed supplements, such as traditional sugar syrup (sucrose), patties, granules, powders, compressed tablets, and the like, such as Mann Lake Ultra Bee Dry Feed. Additional beneficial ingredients, such as amino acids, vitamins (e.g., B complex), fats/lipids, probiotics (e.g., L. acidophilus, E. faecium, B. bifidum, B. licheniformis, B. pumilus, and/or L. plantarum), enzymes/prebiotics, essential oils, yeast, plant polyphenols, phytonutrients, minerals, herbs, proteins, and carbohydrates can be included in the feed or supplement.

Commercial supplements or feed substitutes are available, such as Purina® Hearty Bee™, SuperDFM-HoneyBee, MegaBee®, Nozevit Plus, and the like. Various supplements can be used dry, or mixed with sugar syrup. Supplements and/or feed are also formulated as compressed tablets or patties (e.g., Bee-Pro Patties+, Ultra Bee Patties, etc.), and fed to the bees and/or larvae. Such feed may be used as a supplement or substitute for the honeybees' natural diet, and offered inside the hive or brood box, or freely outside in the vicinity of the hive where bees will otherwise come into contact with the feed. Additional base ingredients used in such feed substitutes often include vegetable oils, such as canola or sunflower oil, and grain flours, such as soy, sorghum, or wheat flour, corn meal, and yeast, such as brewer's yeast. A carbohydrate/sugar source is added to the feed in the form of sucrose, corn syrup, sugar syrup, cane, or beet sugar. Additional inactive binders or excipients can also be included in the composition including cellulose, starches, natural waxes, and the like.

Thus, embodiments of the invention further comprise methods of prophylaxis or treatment of bacterial and/or fungal infections in a bee colony. The antibody composition can be used in conjunction with additional colony treatments for disease and/pests, such as a those containing antibiotics, miticides, formic acid, nutraceuticals (thymol), etc. for honeybee dysentery, mites, parasites, etc. Preferably the antibodies have been purified from chicken eggs, more preferably from hens immunized with a bacterial antigenic agent (e.g., Paenibacillus larvae) or fungal antigenic agent (e.g., Nosema ceranae or Nosema apis). Regardless, the antibody composition is fed to and ingested by the bees and/or larvae at therapeutically-effective dosages and/or timing intervals to prevent and/or treat bacterial and/or fungal infections in the bee colony. As used herein, the term “therapeutically effective” refers to the amount and/or time period that will elicit the biological response in the treated insect or larvae being sought by a researcher or clinician, and in particular elicit protection against bacterial and/or fungal infection symptoms and accordingly reduce mortality. For example, in one or more embodiments, therapeutically effective amounts and time periods are those that deliver an effective amount of antibodies to the bees and/or larvae being treated. One of skill in the art recognizes that an amount or time period may be considered “therapeutically effective” even if the condition is not totally prevented or eradicated but improved partially. Preferably, methods of the invention are effective in reducing incidence of mortality due to bacterial and/or fungal infections by at least 50% in a beehive or colony (50% or less of bees exhibiting signs of bacterial and/or fungal infections), and even more preferably by at least 60%.

### BRIEF DESCRIPTION OF THE DRAWINGS

FIG. 1 is a graph showing the growth inhibition of P. larvae in vitro by anti-P. larvae antibodies.

FIG. 2 is a graph of the cumulative mortality (%) in the larvae from the different experimental groups at day 3 (larvae were fed antibody and challenged with P. larvae on day 0).

### DETAILED DESCRIPTION

The following description set forth methods in accordance with the invention. It is to be understood, however, that these examples are provided by way of illustration and nothing therein should be taken as a limitation upon the overall scope of the invention.

### Generation of Antibodies

In one or more embodiments, antibodies against bacterial infection (e.g., Paenibacillus larvae) are generated by hyperimmunization of chickens with the whole pathogen, killed or attenuated forms thereof, or pathogenic fragments (antigens) thereof (collectively “antigenic agents”). In one or more embodiments, antibodies against fungal infection (e.g., Nosema spp.) are generated by hyperimmunization of chickens with the whole pathogen, killed or attenuated forms thereof, spores, or pathogenic fragments (antigens) thereof (collectively “antigenic agents”). The bacterial and/or fungal antigenic agents are combined with a pharmaceutically-acceptable carrier with optional adjuvant to create the immunizing formulation and used to immunize laying hens. In one or more embodiments, recombinant bacterial or fungal antigens could be used in the immunizing formulation to immunize the hens. As used herein, the term “pharmaceutically acceptable” means not biologically or otherwise undesirable, in that it can be administered to a subject without excessive toxicity, irritation, or allergic response, and does not cause unacceptable biological effects or interact in a deleterious manner with any of the other components of the composition in which it is contained. A pharmaceutically-acceptable carrier would be selected to minimize any degradation of the administered antigens as would be well known to one of skill in the art. In one or more embodiments, the bacterial and/or fungal antigenic agents are combined with an oil-in-water adjuvant such as Freund' s incomplete adjuvant at 50% or Emulsigen (MVP laboratories) at 20% final volume, preferably in aqueous solution (e.g., saline, or buffered solutions such as PBS) to create the immunizing formulation. In one or more embodiments, laying hens are injected with the immunizing formulation biweekly at least twice to initiate a strong initial immune response, and thereafter periodically boosted (e.g., one, two or more times) to maintain the immune response in the hens, resulting in passive generation of antibodies against bacterial and/or fungal infection in eggs laid by the immunized hens.

### Antibody Purification and Quantification

Immunoglobulin Y is purified from egg yolk as previously described (Sun et al., 2018). Antibody titer is determined qualitatively by enzyme linked immunosorbent assay using purified bacterial and/or fungal antigenic agents as antigen coated to the Immulon 2HB polystyrene 96 well plates at 1 ug/well. The purified antibodies as serially diluted two-fold down the 96-well plate and the titer is determined by the reciprocal dilution which yields an absorbance significantly greater than control non-immunized egg yolk antibodies using a secondary horseradish peroxidase (HRP) labeled anti-chicken antibody coupled to a suitable chemical substrate for HRP.

### Commercial Use

A commercial antibody solution is formulated such that it contains a protective titer of antibodies. Bees are routinely fed a commercial sugar solution or supplements at multiple times per year at times of stress such as prior to winter and emergence in spring. The commercial antibody solution is typically composed of water, glucose, antibodies and preservatives. Alternately, the antibodies are lyophilized and sold as a solid composition which is suitably rehydrated with commercially-available bee feed, or use with dry bee feed or supplements. In general, therapeutically-effective dosage amount is determined via challenge of larvae and use of that material as a standard. Subsequent formulations are matched to this concentration using an ELISA. In brief, 96 well polystyrene plates are coated with P. larvae spores and blocked with a protein buffer. A dilution series of therapeutically-effective antibody solution is added to the plate along with a dilution series of the new material to be formulated. Following 1 hr incubation and washing, anti-chicken IgY conjugate is added to all wells and incubated 1 hour. Following washing, the plate is developed using a chromogenic substrate. Absorbance is calculated for all wells and used to plot absorbance as a function of dilution. This allows for relative potency calculation. This information is used to dilute the new material to the concentration of the therapeutically-effective standard.

The bacterial and/or fungal antibody composition is provided prophylactically prior to and post-winter hibernation. The antibody composition is also used therapeutically in response to signs of CCD, namely loss of bees in the colony or colonies exhibiting signs of American foulbrood, or fungal or spore infestation. In addition to the antibodies, the compositions can also be used to deliver nutrients, hormones, or other beneficial agents (e.g., brewer's yeast) to bees and/or larvae in the bee colony. The bacterial and/or fungal antibody composition is placed in a location where the bees and/or larvae will come into contact with (and feed upon) the composition. In one or more embodiments, the composition is placed inside the hive, brood box, or other bee colony enclosure associated with the hive. In one or more embodiments, the composition is positioned outside the hive or brood box, but in the vicinity of the hive where bees will otherwise come into contact with the composition (and transport it back to the hive). The compositions can also be paired with a container or structure for containing and/or slowly releasing the composition, such as a paper strip, foam pad or sponge, diffusor, saucer, and the like.

The bacterial and/or fungal antibody composition can be used in conjunction with additional colony treatments for disease and/pests, such as a those containing antibiotics, miticides, formic acid, nutraceuticals (thymol), etc. for honeybee dysentery, mites, parasites, etc. Additional active agents may be included in the composition or co-administered (as separate compositions) to the bees or placed in, on, or near the hive, including antibiotics, antiparasitics, and mixtures thereof. The compositions can also be used in combination with pesticides. These additional active agents include those not formulated for feeding to the bees and/or larvae, but simply as treatment for the hive environment.

Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the invention. It is to be understood, however, that these examples are provided by way of illustration and nothing therein should be taken as a limitation upon the overall scope of the invention.

### Bacterial Antibodies

Whole inactivated Paenibacillus larvae bacteria were combined with Freund's incomplete adjuvant at 50% final volume and used to inject laying hens biweekly four times. Antibodies were subsequently purified from the egg yolks using published protocols and used for the study.

Briefly, twelve white leghorn chickens, approximately 6-months of age, were immunized four times with the P. larvae vaccine. Each vaccine consisted of 9.0 log₁₀ colony forming units of inactivated P. larvae and 50% incomplete Freund's adjuvant with the remaining volume comprised of phosphate buffered saline. The birds were administered with a 0.5 cc intramuscular injection in each breast. Immunizations were given at 14-day intervals. Eggs were collected daily, beginning two weeks after the third vaccination. Yolks were separated from the albumin using a wire egg separating apparatus. The yolks were then processed according to published protocols. Briefly, yolks were diluted with seven volumes of tap water and brought to a pH of 5.0 using 1.0 N HCl. This was done in 1-gallon containers, with each container consisting of 500 mL yolk, 3 L water, and 36 mL 1.0 N HCl. A pH probe was used to verify the pH of each aliquot. This solution was frozen overnight at −18° C. and thawed the following day. Upon thawing, the solution separated into two distinct layers: an upper aqueous layer (mostly transparent) containing the IgY and a lower viscous layer, containing lipids and other insoluble material (orange). The upper aqueous layer was collected with a pipet while the bottom lipid layer was discarded. The aqueous layer was then concentrated using MiniKros Hollow Fiber Filter Module; P/N: N04-E030-05-N; Media/Rating: mPES/30 k; Surface Area: 5400 cm²; Max. Op. Pressure 30 psig (2 bar); SN: 3303990-07/18-002. The volume of aqueous layer was reduced approximately 10-fold. It was estimated that each egg provides approximately 16.67 mL of yolk which when processed as above, yielded 11.05 mL of concentrated anti-P. larvae antibody.

Growth Inhibition of Paenibacillus larvae In Vitro Using Purified Anti-P. larvae Antibodies

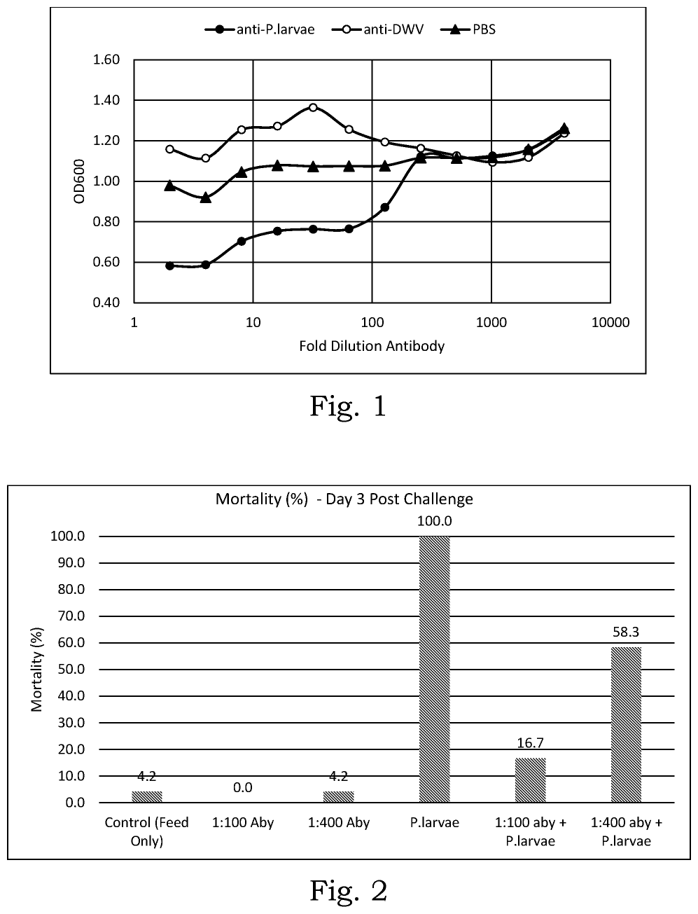

All wells of a polystyrene 96 well microplate were filled with 100 μL of sterile Brain Heart Infusion broth. Next 100 μL of purified anti-P. larvae antibodies were added to duplicate wells in column 1 (wells A1 and B1). As a control, 100 μL of purified anti-deformed wing virus antibodies were added to duplicate wells in column 1 (wells C1 and D1). Phosphate buffered saline, 100 μL, was added to wells E1-H1. Samples in column 1 were mixed by pipetting with a multichannel pipettor and 100 μL was transferred to column 2. This procedure was repeated across the microplate with the final 100 μL removed from column 12 discard. This procedure creates a dilution series of antibodies from 2-fold to 4,096-fold. Next, 100 μL of P. larvae culture in BHI at an optical density 600 nm of 1.0 was added to all wells and the plate was incubated overnight at 37° C. After 18 hours, the plate optical density was read at 600 nm. As can be seen in FIG. 1, P. larvae growth was inhibited by addition of anti-P. larvae antibodies from dilutions 2-128 as evident by decreased OD600 values compared to PBS and anti-DWV controls. At a dilution of 256 and higher, no growth inhibition was observed. These results demonstrate that anti-P. larvae antibodies generated in chickens and purified by the above described methodology are functional and capable of inhibiting P. larvae growth in culture.

### Larval Experiments

In order to test the effectiveness of the antibody, worker bee larvae were grown in vitro according to published protocols. A frame containing at least 25% eggs and 1^{st} instar larvae was selected from a hive and transported to the lab in an insulated box. The frame was kept humidified (i.e., draped in wet paper towels) during transport. One-day old A. mellifera larvae free of bacterial or fungal infection are were grafted from the frame to queen cell cups (Mann Lake Ltd. Cat. #QC-520) inserted into each well 48-well tissue culture plates. Each cup contained 20 μL of Diet A at the time of grafting. Larvae were fed generally according to Schmehl's feeding schedule (see Tables 1-2) with some exceptions. Briefly, larvae were fed 30 μL of Diet A containing treatment (antibody) and challenge (P. larvae) on day 0, post-grafting, for the indicated groups. Larvae were fed an additional 20 μL of diet A on day 2. The experiment endpoints on day 3 so that is all for the feeding. Larvae receiving challenge were administered challenge only on day 0, while larvae receiving antibody received it at the desired concentration at each feeding. Feed was directly pipetted down the side of the cup to avoid drowning the larvae.

TABLE 1
Percent composition of larval feed. Antibody and
challenge were added at desired concentrations.

| | Royal Jelly | Glucose | Fructose | Yeast Extract | Water |
|---|---|---|---|---|---|
| Diet A | 44.25% | 5.30% | 5.30% | 0.90% | 44.25% |

TABLE 2
Feeding schedule of larvae; days correspond to time since grafting.
Antibody groups were fed antibody in each feeding; however, challenge
(in applicable group) was only administered on day 0.

| Day 0 | Day 1 | Day 2 |
|---|---|---|
| 20 μL Diet A | None | 20 μL Diet A |
| 10 μL Diet A with Treatment | | |

The larvae were subjected to various treatment protocols shown in Table 3.

TABLE 3
Treatment groups

| Group | P. larvae Challenge |
|---|---|
| Control (feed only) | No |
| 1:100 anti-P. larvae | No |
| 1:400 anti-P. larvae | No |
| Challenge control | Yes |
| 1:100 anti-P. larvae | Yes |
| 1:400 anti-P. larvae | Yes |

Larvae were then placed in a desiccator with a cup of supersaturated salt solution consisting of 80 g K₂SO₄, 200 g NaCl, and 500 mL H₂O and incubated at 35° C. in a humidified incubator. Mortality assessment and feeding occurred every 24 hours post-grafting. The negative control group received only feed. Two treatment groups received different antibody concentrations (2 dilutions, 1:100 dilution of concentrate, 1:400 dilution of concentrate), without challenge, to investigate the safety of feeding the antibodies to the larvae. The positive control group did not receive any antibodies but was challenged. Two final treatment groups received feed spiked with bacterial antibodies produced in the eggs at different concentrations (2 dilutions, 1:100 dilution of concentrate, 1:400 dilution of concentrate) as noted in the table below. Larvae in these groups were simultaneously challenged with P. larvae by oral administration.

Larvae were monitored for 3 days post challenge and dead larvae were quantified. All dead larvae were removed daily, sorted by treatment group, and frozen for further analysis. Larvae from each treatment group were separated into “live” and “dead” pools. Larvae were considered “live” only if they survived until the experiment's endpoint.

As shown in FIG. 2, the minimum protective dose was determined statistically as the titer of antibody required to significantly improve bacterial and/or fungal infection-associated mortality as compared to the no-antibody control.

### Results

As can be seen, the antibodies appeared generally safe, particularly at low levels. Further, administration of the antibodies significantly improved survival. By day 3, 100% (24/24) of larvae challenged with P. larvae had died, compared to only 16.7% (4/24) of larvae fed anti-P. larvae at 1:100 dilution. Higher mortality, 58.3% (14/24), was observed for larvae fed a lower concentration (1:400 dilution) of anti-P. larvae. These results demonstrate that anti-P. larvae antibodies are capable of reducing mortality by over 80%. Additionally, the high efficacy of 1:100 dilution of anti-P. larvae is consistent with in vitro growth inhibition studies which demonstrated that a similar dilution (1:128) was capable of inhibiting P. larvae growth in vitro.

The outcome of this research is surprising and exciting. Given that insects only have rudimentary immune systems and no adaptive immunity, the use of antibodies as a treatment protocol for preventing or treating bacterial and/or fungal infection represents as significant advancement in the fight to reduce CCD and save honeybee populations.

The same process is followed to generate antibodies against Nosema ceranae. These experiments are similar to the above protocol, but instead of challenging with P. larvae we would challenge with Nosema ceranae spores and feed dilutions of anti-Nosema antibodies.

1. A prophylactic and/or therapeutic food composition for control of bacterial and/or fungal infection in bees and/or bee larvae, said composition comprising bacterial and/or fungal antibodies dispersed in an edible base composition.

2. The food composition of claim 1, wherein said edible base composition is selected from the group consisting of a liquid solution, dry granules or powder, a compressed tablet, and a patty.

3. The food composition of claim 1, wherein said edible base composition comprises a carbohydrate source or is formulated for mixing with a carbohydrate source.

4. The food composition of claim 3, wherein said carbohydrate source is sucrose, corn syrup, sugar syrup, cane, or beet sugar.

5. The food composition of claim 1, wherein said composition further comprises one or more of amino acids, vitamins, fats/lipids, probiotics, enzymes/prebiotics, essential oils, yeast, plant polyphenols, phytonutrients, minerals, herbs, and proteins.

6. The food composition of claim 1, wherein said composition further comprises one or more antibiotics, miticides, formic acid, nutraceuticals, or combinations thereof.

7. The food composition of claim 1, wherein said bacterial and/or fungal comprise anti-Paenibacillus larvae or Nosema spp. antibodies.

8. The food composition of claim 1, wherein said bacterial and/or fungal antibodies are IgY antibodies.

9. The food composition of claim 8, wherein said IgY antibodies are purified from chicken eggs from hens immunized with bacterial and/or fungal antigenic agents, wherein said IgY antibodies are specific for said bacterial and/or fungal antigenic agents.

10. A method of prophylaxis or treatment of bacterial and/or fungal infection in a bee colony, said method comprising: placing a composition according to claim 1 in a location where a bee or bee larvae from said colony will come into direct contact with said composition. inside a bee colony enclosure, such as a hive or brood box, or

11. The method of claim 10, wherein said composition is placed inside a bee colony enclosure, beehive, or brood box.

12. The method of claim 10, wherein said composition is placed outside a bee colony enclosure proximate to the enclosure where bees will otherwise come into contact therewith.

13. The method of claim 10, wherein said composition is ingested by said bee or bee larvae at a therapeutically-effective dosage or over a therapeutically-effective time interval.

14. The method of claim 10, wherein said method is effective in preventing bee colony collapse disorder.

15. The method of claim 10, wherein said method is effective in treating bee colony collapse disorder.

16. The method of claim 10, wherein bee colony is free of observable signs of bacterial and/or fungal infection prior to said placing of said composition in a location where a bee or bee larvae from said colony will come into direct contact with said composition.

17. The method of claim 10, wherein said bee colony shows observable signs of bacterial and/or fungal infection prior to said placing of said composition in a location where a bee or bee larvae from said colony will come into direct contact with said composition.

18. A method of preparing a prophylactic and/or therapeutic food composition for control of bacterial and/or fungal infection in bees and/or bee larvae, said method comprising:
immunizing hens with bacterial and/or fungal antigenic agents;
purifying bacterial and/or fungal antibodies from eggs laid by said immunized hens, and
combining said bacterial and/or fungal with an edible base composition for bees to yield said prophylactic and/or therapeutic food composition for control of bacterial and/or fungal infection in bees and/or bee larvae.

19. The method of claim 18, further comprising providing said composition in or near a beehive such that bees and/or larvae come into direct contact therewith for prophylaxis or treatment of bacterial and/or fungal infection in the bees and/or larvae.

20. (canceled)
